(19) United States
(12) Patent Application Publication
(10) Pub. No.: US 2013/0172621 A1
(43) Pub. Date: Jul. 4, 2013

(54) INTEGRATED PROCESSES FOR THE PREPARATION OF POLYBENZIMIDAZOLE PRECURSORS

(71) Applicants: Rajiv Dhawan, Wilmington, DE (US); Annalisa Hargis, Wilmington, DE (US); Joachim C. Ritter, Wilmington, DE (US)

(72) Inventors: Rajiv Dhawan, Wilmington, DE (US); Annalisa Hargis, Wilmington, DE (US); Joachim C. Ritter, Wilmington, DE (US)

(73) Assignee: E.I.Du Pont de Nemours and Company, Wilmington, DE (US)

(21) Appl. No.: 13/517,272

(22) PCT Filed: Dec. 21, 2010
(86) PCT No.: PCT/US10/61525

§ 371 Date: Aug. 8, 2012

Related U.S. Application Data
(60) Provisional application No. 61/288,477, filed on Dec. 21, 2009.

(51) Int. Cl. C07C 213/02

(52) U.S. Cl. CPC C07C 213/02
USPC 564/443

(57) ABSTRACT

An integrated process is provided for efficiently preparing 2,4,5-triaminophenol, starting with nitration of 2,6-dihalobenzene; high purity salts thereof; and complexes of 2,4,5-triaminophenol aromatic diacids, which are precursors for making polybenzimidazole polymer for high performance fibers. The process design eliminates several costly intermediate drying and recrystallization steps. The handling of solid materials with possible skin sensitizing properties and toxicity is avoided, thereby eliminating human and environmental exposure.

This application claims priority under 35 U.S.C. §119(e) from, and claims the benefit of, U.S. Provisional Application No. 61/288,477, filed Dec. 21, 2009, which is by this reference incorporated in its entirety as a part hereof for all purposes.

### FIELD OF DISCLOSURE

This disclosure relates to methods of making 2,4,5-triaminophenol and salts and complexes thereof, with can be used to make high-performance polybenzimidazole polymers.

### BACKGROUND

Aromatic amines and phenols are useful monomers for high performance polymers such as aramid polymers and polybenzimidazoles. Monomer structure affects both finished article properties, such as fiber tenacity, and the rheological behavior of the polymer during processing such as spinning. Asymmetric monomers, as opposed to highly symmetric ones such as 1,2,4,5-tetraamonobenzene, are desired to increase the solubility of the corresponding polymers for improved fiber spinning. The synthesis of the preferred polybenzimidazole-based high performance fibers then requires the selective polymerization of an asymmetric monomer, such as 2,4,5-triaminophenol (“TAPH”), with various substituted and unsubstituted aromatic diacids, such as 2,5-dihydroxyterephthalic acid (“DHTA”). However, no synthetic route has been identified for making TAPH and related compounds.

There remains a need for a process for the safe and efficient production of high-purity 2,4,5-triaminophenol (TAPH), and salts of 2,4,5-triaminophenol that can be converted to 2,4,5-triaminophenol, to make an aromatic diacid complex of 2,4,5-triaminophenol of high enough purity for use in making a high molecular weight polymer material for producing high-performance fibers. For reasons of cost and safety, it would be highly desirable to have a process where intermediates do not need to be isolated as dry materials.

### SUMMARY

In one embodiment, the inventions hereof provide a process comprising the sequential steps under the substantial exclusion or exclusion of oxygen:
(a) nitrating 1,3-dihalobenzene (II)

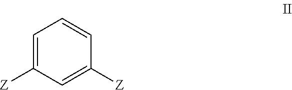

wherein each Z is independently Cl or Br, comprising contacting 1,3-dihalobenzene in a reaction mixture comprising with oleum or SO₃, nitric acid, and H₂SO₄
wherein
   (i) the concentration of nitric acid is about 2.0 to about 2.3 moles per mole of 1,3-dihalobenzene;
   (ii) the concentration of SO₃ is about 1 to about 3 moles per mole of 1,3-dihalobenzene;
   (iii) the concentration of 1,3-dihalobenzene in the reaction mixture is between about 12 and about 24 weight percent; and
      wherein the temperature of the reaction mixture does not exceed 120° C.; thereby producing 1,3-dihalo-4,6-dinitrobenzene (III);

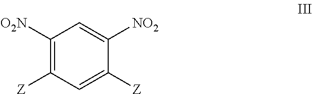

(b) separating the 1,3-dihalo-4,6-dinitrobenzene from the reaction mixture, while recycling the sulfuric acid mother liquor;
(c) washing the 1,3-dihalo-4,6-dinitrobenzene with water or acid then water, then with aqueous ammonia, and then mixing it with solvent as a suspension;
(d) monoaminating the 1,3-dihalo-4,6-dinitrobenzene by heating the suspension formed in step (c) to a temperature in the range of about 60° C. to about 140° C. and contacting it with at least 2.0 equivalents NH₃, thereby converting the 1,3-dihalo-4,6-dinitrobenzene to 1-amino-3-halo-4,6-dinitrobenzene (IV);

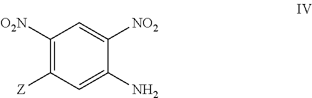

(e) separating the 1-amino-3-halo-4,6-dinitrobenzene from the reaction mixture, washing with solvent, then washing with water;
(f) forming a slurry of the 1-amino-3-halo-4,6-dinitrobenzene with benzyl alcohol and at least 1.0 equivalent of NaOH or of sodium benzyloxide; thereby converting the 1-amino-3-halo-4,6-dinitrobenzene to 1-benzyloxy-3-amino-4,6-dinitrobenzene (V);

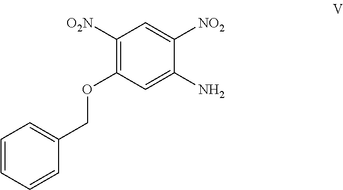 [SMILES: Nc1cc(OCc2ccccc2)c([N+](=O)[O-])cc1[N+](=O)[O-]]

(g) separating the 1-benzyloxy-3-amino-4,6-dinitrobenzene formed in step (f) from the reaction mixture;
(h) forming a slurry of the 1-benzyloxy-3-amino-4,6-dinitrobenzene formed in step (f) with water and transferring the slurry to a hydrogenation reactor containing a hydrogenation catalyst to form a reaction mixture;
(i) hydrogenating the 1-benzyloxy-3-amino-4,6-dinitrobenzene in water by contacting the reaction mixture formed in step (h) with hydrogen at a pressure in the range of about 0.31 to about 3.45 MPa and a temperature in the range of about 20° C. to about 100° C. for sufficient time to hydrogenate the 1-benzyloxy-3-amino-4,6-dinitrobenzene, thereby producing 2,4,5-triaminophenol and toluene;
(j) contacting the reaction mixture (i) with an aqueous solution comprising 1 to 2 equivalents of acid per mol of 2,4,5-triaminophenol and, optionally, heating the solution, thereby dissolving the 2,4,5-triaminophenol;
(k) removing the spent hydrogenation catalyst from the reaction mixture;
(l) extracting toluene from the reaction mixture;
(m) adjusting the pH of the extracted reaction mixture to a value between about 5 and about 7, by adding a base wherein said base does not increase 2,4,5-triaminophenol solubility, thereby precipitating 2,4,5-triaminophenol product; and
(n) isolating the 2,4,5-triaminophenol product.

### BRIEF DESCRIPTION OF THE DRAWINGS

The invention is illustrated by way of example and not limitation in the accompanying figures.

FIG. 1 is a schematic representation of an embodiment of the process described herein for preparing TAPH and TAPH salt.

FIG. 2 is a schematic representation of an embodiment of the process described herein for preparing TAPH complex.

### DESCRIPTION

The following description is exemplary and explanatory only and is not restrictive of the invention, as defined in the appended claims.

In one embodiment of this invention, a process, which may be an integrated process, comprising the sequential steps under the substantial exclusion or exclusion of oxygen:
(a) nitrating 1,3-dihalobenzene (II)

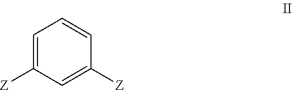

wherein each Z is independently Cl or Br, by contact with a reaction mixture comprising oleum or SO₃, nitric acid, and H₂SO₄
wherein
   (i) the concentration of nitric acid is about 2.0 to about 2.3 moles per mole of 1,3-dihalobenzene;
   (ii) the concentration of SO₃ is about 1 to about 3 moles per mole of 1,3-dihalobenzene,
   (iii) the concentration of 1,3-dihalobenzene in the reaction mixture is between about 12 and about 24 weight percent; and
wherein the temperature of the reaction mixture does not exceed 120° C.;
thereby producing 1,3-dihalo-4,6-dinitrobenzene (III);

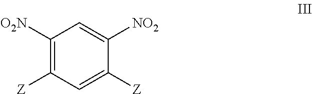

(b) separating or directly separating the 1,3-dihalo-4,6-dinitrobenzene from the reaction mixture, such as by filtration, while recycling the sulfuric acid mother liquor;
(c) washing the 1,3-dihalo-4,6-dinitrobenzene with water or acid then water, then with aqueous ammonia, and then mixing it with solvent as a suspension;
(d) monoaminating the 1,3-dihalo-4,6-dinitrobenzene by heating the suspension formed in step (c) to a temperature in the range of about 60° C. to about 140° C. and contacting it with at least 2.0 equivalents NH₃, thereby converting the 1,3-dihalo-4,6-dinitrobenzene to 1-amino-3-halo-4,6-dinitrobenzene (IV);

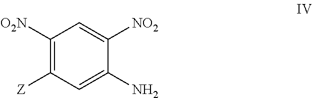

(e) separating or directly separating the 1-amino-3-halo-4,6-dinitrobenzene from the reaction mixture, such as by filtration, washing with solvent, then washing with water;
(f) forming a slurry of the 1-amino-3-halo-4,6-dinitrobenzene with benzyl alcohol and at least 1.0 equivalent of NaOH or of sodium benzyloxide; thereby converting the 1-amino-3-halo-4,6-dinitrobenzene to 1-benzyloxy-3-amino-4,6-dinitrobenzene (V);

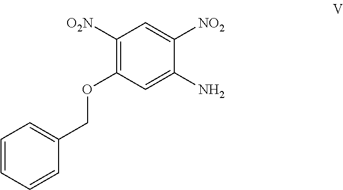 [SMILES: Nc1cc(OCc2ccccc2)c([N+](=O)[O-])cc1[N+](=O)[O-]]

(g) separating or directly separating the 1-benzyloxy-3-amino-4,6-dinitrobenzene formed in step (f) from the reaction mixture, such as by filtration;
(h) forming a slurry of the 1-benzyloxy-3-amino-4,6-dinitrobenzene formed in step (f) with water and transferring the slurry to a hydrogenation reactor containing a hydrogenation catalyst to form a reaction mixture;
(i) hydrogenating the 1-benzyloxy-3-amino-4,6-dinitrobenzene in water by contacting the reaction mixture formed in step (h) with hydrogen at a pressure in the range of about 0.31 to about 3.45 MPa and a temperature in the range of about 20° C. to about 100° C. for sufficient time to hydrogenate the 1-benzyloxy-3-amino-4,6-dinitrobenzene, thereby producing 2,4,5-triaminophenol and toluene;
(l) contacting the reaction mixture (i) with an aqueous solution comprising 1 to 2 equivalents of acid per mol of 2,4,5-triaminophenol and, optionally, heating the solution, thereby dissolving the 2,4,5-triaminophenol;
(k) removing the spent hydrogenation catalyst from the reaction mixture, such as by filtration;
(l) extracting toluene from the reaction mixture;
(m) adjusting the pH of the extracted reaction mixture to a value base does not increase 2,4,5-triaminophenol solubility, thereby precipitating 2,4,5-triaminophenol product; and
(n) isolating the 2,4,5-triaminophenol product, such as by filtration.

In a second embodiment, an integrated process for preparing 2,4,5-triaminophenol salt comprises steps (a) through (n) and further comprises slurrying or dissolving the 2,4,5-triaminophenol product in water; adding an acid to the slurry to form and precipitate 2,4,5-triaminophenol salt; and cooling, filtering, and washing the precipitated 2,4,5-triaminophenol salt.

In a third embodiment, an integrated process is provided for preparing a complex of 2,4,5-triaminophenol and an aromatic diacid HOOC-Q-COOH, wherein the complex is generally described by Formula VI,

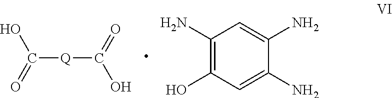

wherein Q is a C₆˜C₂₀ monocyclic or polycyclic aromatic nucleus, by the above described process for preparing the 2,4,5-triaminophenol salt, further comprising slurrying the washed product in water, and adding a base such as NaOH or KOH and a diacid source to form the complex.

In a further embodiment, the complex is prepared by directly contacting the filtered, extracted reaction mixture formed in step (1) with a base, such as NaOH or KOH, and a diacid source, to form the complex. In yet another embodiment, the TAPH free base precipitated in step (m) can then be dissolved in about 1-2 equivalents of acid (e.g., HCl) and the solution so produced contacted with a base such as NaOH or KOH and a diacid source to form the complex.

A reducing agent such as tin powder (Sn) may be added to TAPH, TAPH salt, or TAPH complex at various points in the process to prevent or reverse oxidation to corresponding imines or iminoquinoides.

In the description of the subject matter of this application, the following definitional structure is provided, and, unless indicated to the contrary, is to be applied to the following terminology as employed herein:

As used herein, the term “TAPH” or, equivalently, “TAPH free base” denotes the compound 2,4,5-triaminophenol (Formula I)

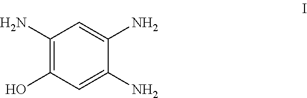

As used herein, the term “TAPH salt” or, equivalently, “2,4,5-triaminophenol salt,” or “TAPH.nA” denotes a compound formed by reaction of 2,4,5-triaminophenol (“TAPH”) with “n” equivalents of an acid (“A”) such as HCl, acetic acid, H₂SO₄, or H₃PO₄. One example of a TAPH salt is TAPH.2HCl (n=2, A=HCl). The salt may also be a hydrate; one such example is TAPH.3HCl.xH₂O.

As used herein the term “diacid source” refers to the diacid HOOC-Q-COOH itself, a disodium salt of HOOC-Q-COOH, a dipotassium salt of HOOC-Q-COOH, or mixtures thereof.

As used herein, the term “XYTA” denotes 2-X-5-Y-terephthalic acid, where X and Y each independently selected from the group consisting of H, OH, SH, SO₃H, methyl, ethyl, F, Cl, and Br. One example is 2,5-dihydroxyterephthalic acid, in which X═Y═OH. The disodium or dipotassium salt of the diacid is represented by the term “M₂XYTA” where M is Na or K.

As used herein, the term “oleum” denotes fuming sulfuric acid, which is anhydrous and is formed by dissolving excess sulfur trioxide (SO₃) into sulfuric acid.

As used herein, the term “fuming nitric acid” denotes concentrated nitric acid containing dissolved nitrogen dioxide.

As used herein, the term “net yield” of P denotes the actual, in-hand yield, i.e., the theoretical maximum yield minus losses incurred in the course of activities such as isolating, handling, drying, and the like.

As used herein, the term “purity” denotes what percentage of an in-hand, isolated sample is actually the specified substance.

The processes are designed in such a way that solids handling is avoided. Filtered materials are transferred, without prior drying, in the form of suspension slurries in the solvent that is used for the respective reaction step. This process design thereby avoids costly drying processes. It also avoids the handling of solid materials with possible skin sensitizing properties and toxicity, and eliminates human and environmental exposure to them.

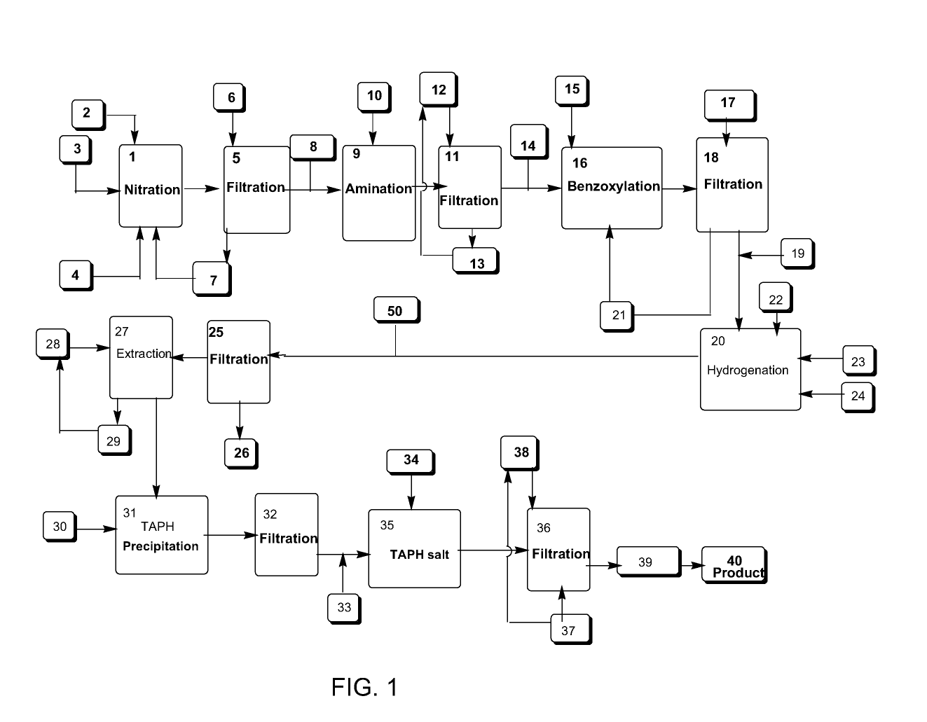

An embodiment of the process described herein to make TAPH free base or TAPH salt is illustrated in FIG. 1; possible minor modifications will be evident to one skilled in the art. With reference to the embodiment shown schematically in FIG. 1, the process starts with the nitration 1 of 1,3-dihalobenzene (i.e., 1,3-dichlorobenzene, 1,3-dibromobenzene, or 1-bromo-3-chlorobenzene, 1,3-dichlorobenzene is preferred), in a reaction mixture prepared by combining the 1,3-dihalobenzene 2, oleum 3, and nitric acid 4. The concentration of nitric acid is about 2.0 to about 2.3 moles per mole of 1,5-dihalobenzene. Concentrated nitric acid (e.g., commonly used reagent grade, which is about 70% nitric acid in water) can be used, but fuming nitric acid is preferred.

If concentrated nitric acid is used, since in the process described herein water must be kept at a level below one equivalent to get highly pure product, more SO₃ would be added to remove the water from the nitric acid (by reacting with it to form sulfuric acid) and still have sufficient SO₃ present in the reaction mixture for the nitration reaction. The concentration of SO₃ is about 1 to about 3 moles, preferably 1.5 to 2 moles, per mole of 1,3-dihalobenzene. The sulfuric acid is present in an amount such that the weight percent of 1,3-dihalobenzene in the reaction mixture (i.e., the weight of 1,3-dihalobenzene relative to the combined weight of 1,3-dihalobenzene plus the acid solution) is between 12 and 24 weight percent.

The nitration reaction is carried out at a temperature not to exceed about 120° C., typically in the range of about 5° C. to about 100° C., preferably in the range of about 5° C. to about 40° C., and more preferably in the range of about 5° to about 15° C. The 1,3-dihalo-4,6-dinitrobenzene thereby produced is separated directly by filtration 5 from the reaction mixture as a crude crystal cake without quench or recrystallization steps. The crude crystal cake is washed (6) with water. Aqueous waste is discarded. The sulfuric acid mother liquor is recycled 7, with a purge drawn to prevent excess sulfuric acid accumulation. The resulting wet cake of 1,3-dihalo-4,6-dinitrobenzene is then mixed with solvent 8 and introduced into the amination reactor 9 as a suspension. A solvent suitable for use includes an organic solvent inert to the reaction such as an aliphatic dihydric alcohol such as ethylene glycol (“glycol”).

The suspension is heated to a temperature in the range of about 60° C. to about 140° C., preferably about 130° C., to dissolve the 1,3-dihalo-4,6-dinitrobenzene in the solvent. The resulting solution is contacted at that temperature with aqueous ammonia in solvent (e.g., glycol) 10 for approximately two to four hours close to ambient pressure; the ammonia solution is fed as it is consumed, as indicated by any convenient analytical technique (e.g., pH monitoring or gaseous ammonia flow rate). At least 2, preferably about 2.03 to about 2.07, equivalents of ammonia are required. At reaction completion, the 1-amino-3-halo-4,6-dinitrobenzene (“AHDNB”) thereby produced can be directly isolated from the reaction mixture since it is only sparingly soluble in aliphatic dihydric alcohol such as glycol at temperatures below 50° C.; impurities remain in solution, and net yields of 85% have been found at greater than 98% purity for 1-amino-3-chloro-4,6-dinitrobenzene specifically. The AHDNB is filtered 11, typically at about 60° C., and washed with solvent or water 12. The mother liquor (filtrate) is collected 13, and the solvent is distilled and recycled; purges are drawn to prevent accumulation.

The wet cake of 1-amino-3-halo-4,6-dinitrobenzene is slurried with benzyl alcohol 14. About one to about two equivalents of base (e.g., NaOH as a slurry in benzyl alcohol, or a solution of the sodium salt of benzyl alcohol, Na—O—CH₂-Ph, also known as sodium benzyloxide) are added 15. The 1-benzyloxy-3-amino-4,6-dinitrobenzene (“BOB”) product thereby produced 16 is mixed with cold (e.g., about 10° C. to about 30° C.) methanol/water (e.g., a 50:50 mixture of methanol and water by volume) 17, isolated by filtration 18, slurried with water 19, and transferred to the hydrogenation reactor 20 as a suspension. Remaining benzyl alcohol is recycled 21.

The hydrogenation reactor also contains a hydrogenation catalyst 22. Examples of suitable hydrogenation catalysts include without limitation Pd/C and Pt/C and mixtures thereof, optionally containing other metals from Groups VIII through X such as Fe. The groups are as described in the Periodic Table in Advanced Inorganic Chemistry by F. A. Cotton and G. Wilkinson, Interscience New York, 2nd Ed. (1966). Of these, Pt/C, and Pd/C, e.g., 10% Pt/C and 10% Pd/C, are preferred. The catalyst is typically used in the amount of about 0.5 to about 5.0 wt % metal based on 1-benzyloxy-3-amino-4,6-dinitrobenzene.

The hydrogenation reactor is purged with nitrogen and then hydrogen. Deaerated water 23 is then added to the reactor. The aqueous suspension is contacted with hydrogen 24 to form a reaction mixture. The reaction is carried out at a temperature in the range of about 20° C. to about 100° C., preferably about 60° C. to about 85° C., and a hydrogen pressure of about 45 to about 500 psi (0.31 to 3.45 MPa) preferably about 300 psi (2.07 MPa). Reaction continues for a time sufficient to consume about 6.5 to 7.5 mol equivalents of hydrogen, thereby producing 2,4,5-triaminophenol (“TAPH”). The time required depends on the details of the specific set up but is typically about 2 hours.

About 1 to about 2 equivalents of acid (e.g., HCl) is added 50 to the reaction mixture to dissolve the TAPH. The resulting reaction mixture is filtered 25, typically at a temperature in the range of about 60° C. to about 80° C., to remove the spent hydrogenation catalyst preferably by passing through a carbon filter bed. The spent catalyst can then be recycled 26.

The filtered reaction mixture is then extracted 27, e.g., with hexanes 28, to remove the toluene produced by the hydrogenation of the 1-benzyloxy-3-amino-4,6-dinitrobenzene. The hexanes can then be recycled 29.

The TAPH free base can then be formed from the aqueous phase of the reaction mixture remaining after filtration and extraction, by addition of base 30 (e.g., NaOH or KOH) to adjust the pH to about 5 to about 7, thereby precipitating the TAPH free base 31. The TAPH free base can then be isolated by filtration, washed, and dried if so desired.

Alternatively, to make the TAPH salt, TAPH.nA, as in the embodiment shown in FIG. 1, the TAPH free base is filtered 32, slurried with water 33, and then contacted with acid “A” 34 to form and precipitate TAPH salt 35. The acid is added at a temperature in the range of about 10° C. to about 80° C. The amount of acid needed for this step will depend on the concentration of TAPH in the filtrate and is readily determined by one skilled in the art. Typically, about 6 to about 8 equivalents of acid (as for example, 38% HCl_{aq}) are needed in this step to precipitate the TAPH salt (for example, as TAPH.2HCl) in about 90% yield. The use of gaseous acid, such as gaseous HCl, might reduce the total volume of liquid needed since the additional introduction of water with aqueous acid in both addition steps increases the absolute solubility of the TAPH salt in the filtered reaction mixture. The addition of equivalent amounts of acid in the gas phase instead of as an aqueous solution (for example, HCl_{gas} instead of HCl_{aq}) may be also desirable since the liquid volumes are thereby reduced, and crystallization yields are expected to be higher as a consequence. More commonly, however, aqueous acid (for example, 30-38 wt % HCl) is used because it is easier to handle than the acid in the gas phase. Aqueous acid can be recovered, distilled, and recycled or used in the acid wash step 37 of the process.

To facilitate the precipitation of the TAPH salt (for example, as TAPH.2HCl) an aliphatic alcohol co-solvent may optionally be added. Examples of suitable alcohol co-solvents included without limitation: methanol, ethanol, n-propanol, and isopropanol.

The reaction mixture containing the precipitated TAPH salt 35 is then cooled to about 5° C. to about 15° C. and stirred, then filtered 36. The TAPH salt is then washed 37. It may be washed with deaerated aqueous acid, such as HCl (33%), which can be recycled 38, and then optionally with deaerated ethanol or methanol to produce a wet cake material. The optional ethanol or methanol wash can then be recycled, and a purge is drawn to prevent accumulation. Using an agitated filter unit during the wash procedures can allow for a reduction of the wash volumes. Under such circumstances, using small amounts of cold (e.g., about 5° C.) water instead of the aqueous acid would be effective; cold water would be used because of lower solubility of the TAPH salt in cold water versus, e.g., room temperature.

Whether aqueous acid or cold water is used as a wash, it may be possible to eliminate the ethanol or methanol wash and dry directly from aqueous wet cake or simply use the wet cake in subsequent processing. It is likely that in a commercial process one would only wash with HCl, and, if desired, dry directly.

The resulting wet cake material (TAPH salt) can be used in subsequent processing without drying or can be dried, as in FIG. 1 39, for example at a pressure less than 400 Torr and a temperature of about 30° C. to about 50° C., under a stream of N₂. The dried product 40 is preferably kept under nitrogen.

The yield of TAPH salt can be increased by recovered additional TAPH salt from the filtrate remaining from the reaction mixture that contained the precipitated TAPH salt (i.e., the “mother liquor”) by, e.g., evaporation of water.

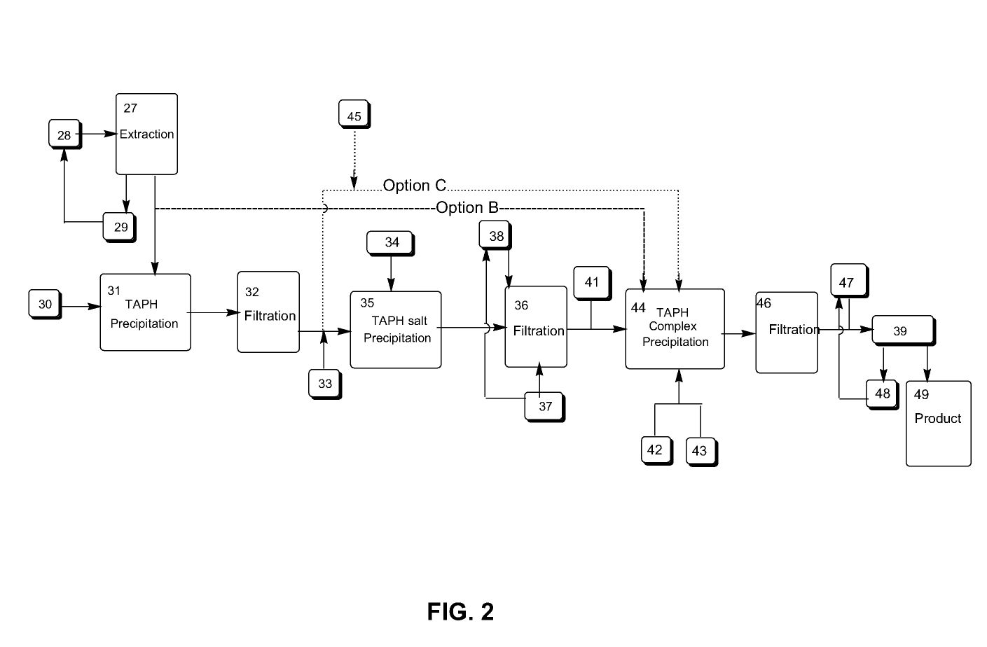

An embodiment of an integrated process to produce the TAPH complex with HOOC-Q-COOH is illustrated in FIG. 2. The diacid HOOC-Q-COOH is an aromatic diacid, wherein Q is a C₆˜C₂₀ substituted or unsubstituted monocyclic or polycyclic aromatic nucleus. Examples of Q include without limitation:

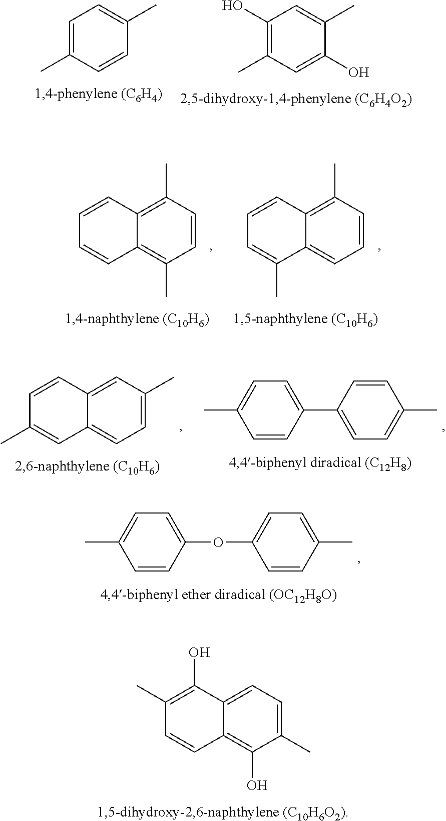

One or more heteroatoms (such as N, O, S) may be present in the ring(s) of Q, for example, as shown below:

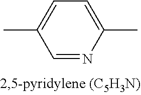

In one embodiment, Q is represented by the structure of Formula (XVIII)

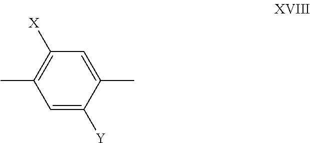

wherein X and Y are each independently selected from the group consisting of H, OH, SH, SO₃H, methyl, ethyl, F, Cl, and Br. Preferably, X═Y═OH (i.e., the diacid is 2,5-dihydroxyterephthalic acid) or X═Y═H (i.e., the diacid is terephthalic acid). When X═Y═H, the diacid is referred to as “XYTA”.

To achieve high productivity in the complex formation process, the TAPH complex can be directly formed from the dissolved TAPH with a disodium or dipotassium salt of the aromatic acid (for example, “M₂XYTA”, wherein M is K or Na) in an aqueous reaction solution.

One embodiment of the process described here is illustrated in FIG. 2; possible minor modifications will be evident to one skilled in the art. In this embodiment, the steps from starting with nitration of 1,3-dihalobenzene through extraction of the reduced, filtered reaction mixture with, e.g., hexanes to remove toluene, are the same as shown in FIG. 1; therefore, FIG. 2 shows the process steps from the extraction of toluene (27, 28, 29) onward.

With reference to an embodiment shown in FIG. 2, herein referred to as “Option A,” the TAPH salt is precipitated and washed as described previously (30 through 38), then slurried with or dissolved water 41. Base (e.g., NaHCO₃) sufficient to neutralize the reaction mixture 42 and the diacid source 43 are then added to the slurry to form and precipitate the TAPH complex 44 (Formula VI).

Alternatively, the extracted, filtered reaction mixture can be combined directly with the base 42 and the diacid source 43 to form and precipitate the TAPH complex 44, as indicated by the dashed line labeled “Option B” on FIG. 2. The amount of base needed will depend on how much acid 50 was added to dissolve TAPH before filtering. In another alternative, indicated by the dotted line labeled “Option C” on FIG. 2, filtered TAPH free base 32 can be dissolved in about 1-2 equivalents of acid (e.g., HCl) 45 and the solution so produced contacted with base (e.g., NaOH or KOH) and the diacid source to form the complex 44.

Various designs are possible for combining the TAPH moiety with the diacid source and base to produce the complex in addition to those shown in FIG. 2. The base 42 and diacid source 43 are most conveniently added as a single solution. In other embodiments, TAPH salt in an acid solution could be introduced into a vessel containing a basic diacid source solution, or the diacid source stream could be fed into the vessel containing the TAPH salt in an acid solution. Alternatively, the diacid source and TAPH salt could be fed concurrently or consecutively into a buffer solution at the desired pH or into a basic solution. Which design is best for a specific situation will be evident to one of skill in the art.

The TAPH complex is recovered from the reaction mixture by filtration 46 at a temperature in of the range of about 5° C. to about 50° C., preferably about 10° C. to about 15° C., and washed 47 with water and methanol, typically at a temperature in the range of about 15° C. to about 40° C., and then dried 39. The methanol is recycled 48, and a purge is drawn to prevent accumulation. The washed and dried TAPH complex 49 is kept under nitrogen to protect it from oxygen. It is of high enough quality and purity to produce polybenzimidazole polymer of high enough molecular weight to make high performance fibers.

The Option A embodiment illustrated in FIG. 2 can produce higher purity TAPH complex than Options B or C. On the other hand, Options B and C have fewer steps, generate less waste and also require less acid (e.g., HCl) and base (e.g., NaOH), thus lessening raw material and handling cost. All three embodiments produce polymer grade material suitable for the manufacture of high-performance fibers.

Oxygen is substantially excluded, and is preferably excluded, throughout all steps of the processes of making TAPH, the TAPH salt, and the complexes. Oxygen is substantially excluded when the oxygen content is low enough that an insignificant or imperceptible amount of impurities are formed during the reaction, and/or when the oxygen content in the reaction is less than about 1,00 ppm, or less than about 500 ppm, or less than about 250 ppm, or less than about 100 ppm, or less than about 50 ppm, or less than about 10 ppm, or less than about 1 ppm. Deaerated water and deaerated acid are used. A small amount of a reducing agent (e.g., about 0.5% tin powder) is optionally added to one or more of aqueous suspensions or aqueous solutions containing TAPH, TAPH salt, or TAPH complex during the process to reduce impurities caused by oxidation and to prevent further impurity formation by that route.

The process described herein is an efficient and effective way to produce TAPH; high purity TAPH salts, such as TAPH.2HCl; and complexes of TAPH with aromatic diacids, such as 2,5-dihydroxyterephthalic acid, which are precursors for making polybenzimidazole polymer for high performance fibers. This process design eliminates costly intermediate drying and recrystallization steps. The recycling of spent catalyst, acids, glycol, and methanol contributes economical and environmental advantages. And, importantly, handling of solid materials with possible skin sensitizing properties and toxicity is avoided, thereby eliminating human and environmental exposure.

The materials, methods, and examples herein are illustrative only and, except as specifically stated, are not intended to be limiting.

### EXAMPLES

This invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions.

All water used was deaerated and de-ionized water. The examples were carried out under exclusion of oxygen.

The meaning of abbreviations is as follows: “ACDNB” means 1-chloro-3-amino-4,6-dinitrobenzene, “BOB” means 1-benzyloxy-3-amino-4,6-dinitrobenzene, “DCDNB” means 1,3-dichloro-4,6-dinitrobenzene, “equiv” means equivalent(s), “g” means gram(s), “GC” means gas chromatography, “¹H-NMR” means proton nuclear magnetic resonance spectroscopy, “h” means hour(s), “L” means liter(s), “LC” means liquid chromatography, “M” means molar, “mL” means milliliter(s), “min” means minutes, “mmol” means millimole(s), “mol” means mole(s), “MPa” means megapascals, “psi” means pounds per square inch, “wt” means weight, and “2X” means two times.

DCDNB was prepared as described in U.S. patent application Ser. No. 12/335,959. Sodium benzyloxide (CAS Reg. No. 20194-18-7) was purchased from the Aldrich Chemical Company, Milwaukee, Wis., USA.

### Example 1

### Preparation of ACDNB from DCDNB

A three-necked flask was equipped with a thermocouple, magnetic stirrer, septa through which a tube was added for the addition of the ammonium hydroxide solution and reflux condenser with gas outlet. The DCDNB (26.2 g) and ethylene glycol (170 g) were added to the flask. The ammonium hydroxide (28% aqueous NH₃) was pumped into the vessel at a rate of 0.607 mL/min at a temperature of 138° C. and the conversion to product was controlled by GC analysis. When the reaction solution showed less than 1% 1,3-dichloro-2,4-dinitrobenzene and no more than 3% of 1,3-diamino-2,4-dinitrobenzene, the ammonium hydroxide feed was stopped. The reaction suspension was allowed to cool to 30° C. and was subsequently filtered. The yellow colored fine crystalline product was washed with two portions of about 50 mL ethylene glycol followed by 2×50 mL water and methanol before it was air dried. The net yield was about 75% and the purity was >97%.

### Example 2

### Preparation of BOB from ACDNB

A three-necked flask was equipped with a thermocouple, magnetic stirrer and reflux condenser with gas outlet. The gas outlet was equipped with a three-way-splitter connecting the outlet to an oil bubbler and an N₂ line. The ACDNB (21 g) and benzyl alcohol (100 mL) were added to the flask and heated to 50° C. while under a N₂ blanket. About 104 mL of a 1 M solution of sodium benzyloxide (1.08 equiv) was added over a period of 1.5 h at 50° C. and stirred for another 1 h at 50° C. Conversion to product was controlled by LC analysis. After reaction completion, the reaction solution was added to 250 mL of a 50% aqueous methanol under vigorous mixing. The solution was filtered and the solid product of light bronze color was further rinsed with another portion of 50:50 methanol and water. After a final rinse with cold methanol, the filter cake was dried. The net yield was about 75% and the purity was >98%. ¹H NMR (d6 DMSO): 8.74 ppm (s, 1H); 8.10 ppm (b, 2H); 7.50-7.30 (m, 5H); 6.75 ppm (s, 1H).

### Example 3

### Preparation of TAPH.2HCl from BOB

A 1 L stirred Hastelloy autoclave was charged with 120 g of BOB and 3.6 g of 10% Pt/C (dry basis, 50% water). The autoclave was purged 10 times with N₂ and 5 times with H₂ at 90 psi (0.62 MPa). Subsequently, 300 mL of deaerated water (purged with N₂ overnight) were added and the mixture was pressurized at 60° C. to 300 psi (2.07 MPa). Hydrogenation was continued for a total time of about 80 min with an approximate uptake of 2.7 moles of H₂ (6.5 equiv). The excess hydrogen was released and the autoclave was cooled to 40° C. and purged twice with N₂, after which 80 g of deaerated HClₐ, (36.3%, by titration) and 175 g of water was added. The mixture was stirred for 1 hour, then passed through a metal CUNO filter to remove catalyst. The autoclave was rinsed with 30 mL of deaerated water. The solution was directly charged into a purged 2-L vessel.

The reaction mixture was extracted with 2×200 mL hexanes and the organic phase was discarded. The aqueous phase was filtered through a filter packed with celite followed by carbon black and sand. About 0.1 g of Sn powder was added to the filtrate. The mixture was neutralized to pH 6 with aqueous sodium hydroxide (50% by wt) and the free base (TAPH) was isolated by filtration. The free base was subsequently combined with water to form a 50% by wt slurry. In a separate flask, 300 g (10 equivalents) of oxygen-free concentrated aqueous HCl (approximately 34% by wt) was cooled to about 5° C. The free base TAPH slurry was added slowly to the stirred cold HCl solution while maintaining a temperature of about 5° C. After stirring for an additional 2 h at 5° C., the TAPH hydrochloride salt was isolated by filtration and washed 2× with about 50 mL methanol and dried. The net isolated yield was 53 g (60% of theory) and the purity was >99%. Elemental analysis: C 33.56%, N 19.23%, H 5.07%, Cl 33.28%. The structural assignment of the product TAPH.2HCl was confirmed by X-Ray structure analysis.

### Example 4

### Preparation of TAPH.DHTA from TAPH.2HCl solution

6.06 g of K₂DHTA (22.08 mmol) along with 2.69 g of sodium bicarbonate (32.02 mmol) was added to a reaction vessel. This was followed by the addition of 75 g of deaerated water and heating to 75° C. About 33.75 g of 0.18 M TAPH.2HCl salt solution (24.3 mmol) made as described in Example 3 was added to another reaction vessel. The hot solution of K₂DHTA was subsequently added dropwise into the TAPH.2HCl salt solution at room temperature, with fast stirring, over a period of 10 minutes, which resulted in precipitation of a light brown solid. This mixture was then cooled to room temperature, with stirring, for 1.5 hours. The mixture was subsequently filtered and washed with ethanol (50 mL). The solid beige product was allowed to dry for 18 hours under vacuum. ¹H-NMR analysis revealed the TAPH:DHTA ratio as being (1.00:1.01).

It is to be appreciated that certain features of the invention which are, for clarity, described above and below in the context of separate embodiments, may also be provided in combination in a single embodiment. Conversely, various features of the invention that are, for brevity, described in the context of a single embodiment, may also be provided separately or in any subcombination. Further, reference to values stated in ranges includes each and every value within that range.

Unless otherwise defined, all technical and scientific terms used herein have the same meaning as commonly understood by one of ordinary skill in the art to which this invention belongs. In case of conflict, the present specification, including definitions, will control.

Although methods and materials similar or equivalent to those described herein can be used in the practice or testing of the present invention, suitable methods and materials are described herein.

Unless stated otherwise, all percentages, parts, ratios, etc., are by weight. When an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises,” “comprising,” “includes,” “including,” “containing,” “characterized by,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

When the term “about” is used in describing a value or an end-point of a range, the disclosure should be understood to include the specific value or end-point referred to. Use of “a” or “an” are employed to describe elements and components of the invention. This is done merely for convenience and to give a general sense of the invention. This description should be read to include one or at least one and the singular also includes the plural unless it is obvious that it is meant otherwise.

What is claimed is:

1. A process comprising the sequential steps under the substantial exclusion of oxygen:
(o) nitrating 1,3-dihalobenzene (II) 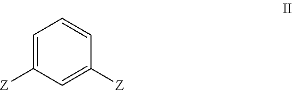
wherein each Z is independently Cl or Br, comprising contacting 1,3-dihalobenzene in a reaction mixture comprising with oleum or SO₃, nitric acid, and H₂SO₄
wherein
(iv) the concentration of nitric acid is about 2.0 to about 2.3 moles per mole of 1,3-dihalobenzene;
(v) the concentration of SO₃ is about 1 to about 3 moles per mole of 1,3-dihalobenzene;
(vi) the concentration of 1,3-dihalobenzene in the reaction mixture is between about 12 and about 24 weight percent; and
wherein the temperature of the reaction mixture does not exceed 120° C.; thereby producing 1,3-dihalo-4,6-dinitrobenzene (III); 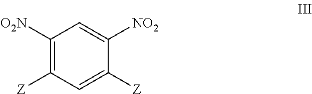
(p) separating the 1,3-dihalo-4,6-dinitrobenzene from the reaction mixture, while recycling the sulfuric acid mother liquor;
(q) washing the 1,3-dihalo-4,6-dinitrobenzene with water or acid then water, then with aqueous ammonia, and then mixing it with solvent as a suspension;
(r) monoaminating the 1,3-dihalo-4,6-dinitrobenzene by heating the suspension formed in step (c) to a temperature in the range of about 60° C. to about 140° C. and contacting it with at least 2.0 equivalents NH₃, thereby converting the 1,3-dihalo-4,6-dinitrobenzene to 1-amino-3-halo-4,6-dinitrobenzene (IV); 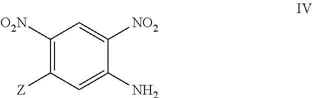
(s) separating the 1-amino-3-halo-4,6-dinitrobenzene from the reaction mixture, washing with solvent, then washing with water;
(t) forming a slurry of the 1-amino-3-halo-4,6-dinitrobenzene with benzyl alcohol and at least 1.0 equivalent of NaOH or of sodium benzyloxide; thereby converting the 1-amino-3-halo-4,6-dinitrobenzene to 1-benzyloxy-3-amino-4,6-dinitrobenzene (V); 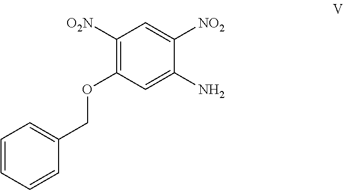 [SMILES: Nc1cc(OCc2ccccc2)c([N+](=O)[O-])cc1[N+](=O)[O-]]
(u) separating the 1-benzyloxy-3-amino-4,6-dinitrobenzene formed in step (f) from the reaction mixture
(v) forming a slurry of the 1-benzyloxy-3-amino-4,6-dinitrobenzene formed in step (f) with water and transferring the slurry to a hydrogenation reactor containing a hydrogenation catalyst to form a reaction mixture;
(w) hydrogenating the 1-benzyloxy-3-amino-4,6-dinitrobenzene in water by contacting the reaction mixture formed in step (h) with hydrogen at a pressure in the range of about 0.31 to about 3.45 MPa and a temperature in the range of about 20° C. to about 100° C. for sufficient time to hydrogenate the 1-benzyloxy-3-amino-4,6-dinitrobenzene, thereby producing 2,4,5-triaminophenol and toluene;
(x) contacting the reaction mixture (i) with an aqueous solution comprising 1 to 2 equivalents of acid per mol of 2,4,5-triaminophenol and, optionally, heating the solution, thereby dissolving the 2,4,5-triaminophenol;
(y) removing the spent hydrogenation catalyst from the reaction mixture;
(z) extracting toluene from the reaction mixture;
(aa) adjusting the pH of the extracted reaction mixture to a value base does not increase 2,4,5-triaminophenol solubility, thereby precipitating 2,4,5-triaminophenol product; and
(bb) isolating the 2,4,5-triaminophenol product.

2. The process of claim 1 further comprising slurrying or dissolving the 2,4,5-triaminophenol product in water; adding an acid to the slurry to form and precipitate 2,4,5-triaminophenol salt; and cooling, filtering, and washing the precipitated 2,4,5-triaminophenol salt.

3. The process of claim 1 wherein the acid in step (j) is selected from the group consisting of HCl, acetic acid, H₂SO₄, and H₃PO₄.

4. The process of claim 3 wherein Z═Cl and the acid in step (j) is HCl.

5. The process of claim 1 wherein the solvent used in step (e) is distilled and recycled.

6. The process of claim 1 wherein the spent hydrogenation catalyst of step (k) is recovered and recycled.

7. The process of claim 2 wherein the acid added to the slurry to form and precipitate 2,4,5-triaminophenol salt is selected from the group consisting of HCl, acetic acid, H₂SO₄, and H₃PO₄.

8. The process of claim 2 wherein the precipitated salt is washed with water and methanol, and the methanol is recycled.

9. The process of claim 2 further comprising drying the 2,4,5-triaminophenol salt.

10. The process of claim 1 further comprising adding a reducing agent to at least one aqueous suspension or aqueous solution containing 2,4,5-triaminophenol or 2,4,5-triaminophenol salt.

11. The process of claim 10, wherein the reducing agent is tin powder.

12. The process of claim 2 further comprising the addition of an aliphatic alcohol co-solvent with the acid to the slurry to form and precipitate 2,4,5-triaminophenol salt.

13. The process of claim 1 wherein the suspension is contacted with 2.03 to 2.07 equivalents NH₃ in step (d).

14. The process of claim 1 wherein the base added in step (m) is NaOH or KOH.

15. The process of claim 1 wherein the toluene is extracted in step (l) with hexanes.
